(19) United States
(12) Patent Application Publication
(10) Pub. No.: US 2009/0118318 A1
(43) Pub. Date: May 7, 2009

(54) Novel Diazaspiroalkanes and Their Use for Treatment of CCR8 Mediated Diseases

(76) Inventors: Stephen Connolly, Leicestershire (GB); Tero Linnanen, Horsholm (DK); Marco Skrinjar, Lund (SE)

Correspondence Address: MORGAN LEWIS & BOCKIUS LLP, 1111 PENNSYLVANIA AVENUE NW, WASHINGTON, DC, 20004, US

(21) Appl. No.: 11/910,579

(22) PCT Filed: Mar. 30, 2006
(86) PCT No.: PCT/SE06/00389

§ 371 Date: Dec. 1, 2008

(30) Foreign Application Priority Data
Apr. 4, 2005 (SE) 0500740-6
Apr. 18, 2005 (SE) 0500873-5

(51) Int. Cl. A61K 31/438; C07D 221/20; A61P 11/00

(52) U.S. Cl. 514/278; 546/16

(57) ABSTRACT

The invention provides compounds of general formula wherein R and R¹ are as defined in the specification, processes for their preparation, pharmaceutical compositions containing them and their use in therapy.

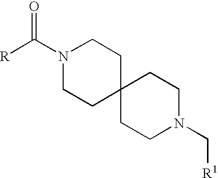

The present invention relates to novel diazaspiro compounds, processes for their preparation, pharmaceutical compositions containing them and their use in therapy.

Both the initial stages of a disease as well as the long-term tissue remodeling and muscle hypotrophy depend on recruitment of leukocytes to the inflammatory lesion. Leukocyte recruitment involves the migration of leukocytes into the diseased tissue from the blood vessel and their activation, which leads to progression of disease. The mechanism underlying this recruitment, chemotaxis, is similar both in classically defined immune mediated pathological conditions (i.e. allergic and autoimmune diseases) as well as others (i.e. atherosclerosis and Parkinson's disease). Thus, intervention of leukocyte recruitment to the inflamed target tissue constitutes an attractive novel therapeutic principle.

The chemokines are a large family (>50 members) of small 8- to 15-kDa secreted, heparin-binding polypeptides with the primary function of controlling trafficking and activation of leukocytes. They are distinct from classical chemoattractants (i.e. bacterial derived N-formyl peptides, complement components, lipid molecules and platelet activating factor) on the basis of shared structural similarities. All chemokines have four conserved cysteines residues that form disulfide bonds, which are critical for the 3-D structure. The chemokines are further subclassed according to the position of the first two cysteines. The two major subclasses are the CC-chemokines, that have the cysteines adjacent, and the CXC-cytokines, that have the cysteines separated by one amino acid. The two other families, the C and the CX3C chemokines, are much smaller and only comprise one or a few members.

The specific biological effects of chemokines, including leukocyte recruitment, are mediated via interactions with a family of seven-transmembrane G-protein coupled receptors (GPCRs). The chemokine receptors are ˜350 amino acids in length and consist of a short extracellular N-terminus, seven transmembrane segments, and an intracellular C-terminus. The seven transmembrane domains are α-helical, and 3 intracellular and 3 extracellular loops exist between the domains.

So far 18 human chemokine receptors have been identified. Of these there are 11 CC chemokine receptors, 5 CXC receptors, 1 CX3C receptor and 1 C receptor. In general, CC chemokines are potent chemoattractants of monocytes and lymphocytes, but poor activators of neutrophils. Certain receptors bind multiple chemokines, for example, CCR1 binds CCL3, CCL5, CCL7 and CCL8, while other chemokine receptors have a more restricted binding profile. This ligand specificity, together with chemokine receptor expression patterns on particular leukocyte subsets, accounts for the regulated, restricted, and specific trafficking of cells into inflammatory lesions. Chemotaxis of inflammatory cells towards a chemokine gradient is initiated by signals mediated by the intracytoplasmatic tail of the chemokine receptor. The downstream signals involve the PI3Kγ, the MAPK and the PKC pathways, among others.

The accumulation of immune cells at a site of allergic inflammation occurs within 6-48 hours after allergen challenge and is a hallmark of allergic diseases. Studies have shown that antigen-specific CD4⁺ T cells are detected in lung tissue in asthmatic patients after exposure to the allergen. Although infiltrating T cells are relatively few in number compared to eosinophils, compelling evidence has demonstrated essential roles for T cells in orchestrating the inflammatory process in human asthma. A close correlation exists in humans between the level of TH2 cytokines produced by T cells, serum level of IgE and prevalence of asthma.

The human CCR8 receptor has been shown to interact with the human chemokine CCL1 (I-309). This chemokine is a potent eosinophil, T cell and endothelial cell chemoattractant. The receptor has been shown to be transiently upregulated on polarized TH2 cells after optimal TCR cross linkage in presence of costimulatory signals (i.e. CD28). The coordinated upregulation of CCR8 on activated T cells after antigen challenge indicates that it contributes to redistribution of the activated T cells to the inflammatory foci within the inflamed tissue expressing CCL1. Indeed, in vivo models of allergic airway inflammation using mice deficient in CCR8 expression have shown a profound block in recruitment of effector T cells to the inflamed lung tissue and production of TH2 cytokines. Moreover, T cells infiltrating the human airway subepithelium during allergen challenge have been shown to be CCR8 positive. Importantly, the number of CCR8 positive cells migrating into the airway submucosa following allergen challenge has been shown to correlate with decreases in FEV1.

Considering the significant role CCR8 plays in TH2 cell chemotaxis, and the importance of TH2 cells in allergic conditions such as asthma, CCR8 represents a good target for drug development in treatment of respiratory diseases, including asthma, chromic obstructive pulmonary disease and rhinitis.

International patent application number WO2005/040167 describes diazaspiro compounds having activity at the CCR8 receptor.

A desirable property for a drug acting at the CCR8 receptor is that it has high potency e.g. as determined by its ability to inhibit the activity of the CCR8 receptor. It is also desirable for such drugs to possess good metabolic stability in order to enhance drug efficacy. Stability against human microsomal metabolism in vitro is indicative of stability towards metabolism in vivo.

The present inventors have identified a set of compounds which show a surprising combination of high potency against CCR8 (determined from inhibition of CCL1 binding to CCR8) and good stability against human microsomal metabolism in vitro.

In accordance with the present invention, there is provided a compound of formula:

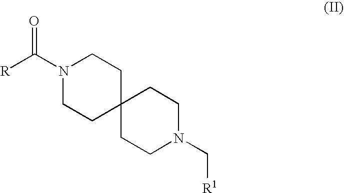

wherein R represents

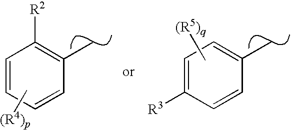

wherein R¹ and R³ independently represent —NR⁸—C(O)—COOH, —O—(C₁₋₄alkyl)-COOH, —C₁₋₄alkyl-COOH, or —COOH;
each R⁴ and R⁵ independently represent halogen, CF₃ or C₁₋₄alkyl;
R⁸ is hydrogen or C₁₋₄alkyl;
p and q are independently 0, 1 or 2;
R¹ represents the group:

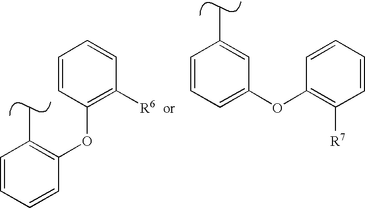

R⁶ and R⁷ are independently hydrogen, methoxy, or ethoxy;
or a pharmaceutically acceptable salt thereof.

The term alkyl, whether alone or as part of another group, includes straight chain and branched chain alkyl groups.

Without being bound to any particular theory, it is believed that the pyridine N-oxide may contribute towards enhancing metabolic stability and the phenoxy-benzyl group on the right hand side of the molecule may contribute towards enhancing CCR⁸ potency.

Compounds with a particularly advantageous combination of high CCR⁸ potency and stability against human microsomal metabolism in vitro were found to be those with R⁶ and R⁷ being methoxy. Thus, in a particularly preferred embodiment, R⁶ and R⁷ are methoxy.

R² and R³ independently represent —NH—C(O)—COOH, —O—(C₁₋₄alkyl)-COOH, —C₁₋₄alkyl-COOH, or —COOH. When R² and R³ represent —O—(C₁₋₄alkyl)-COOH, or —C₁₋₄alkyl-COOH, preferable groups are —O—CH₂—COOH, —O—(CH₂)₂—COOH, —CH₂COOH or —(CH₂)₂—COOH.

In an embodiment of the present invention, R² and R³ independently represent —O—CH₂—COOH, or —CH₂—COOH.

In a further embodiment of the present invention, R² and R³ independently represent —CH₂—COOH.

Each R⁴ and R⁵ independently represent halogen (e.g. chlorine, fluorine or bromine), CF₃ or C₁₋₄alkyl (such as methyl). Preferably, R⁴ and R⁵ are halogen, more preferably chlorine or bromine. In an embodiment of the present invention, R⁴ and R⁵ are chlorine.

p and q are independently 0, 1 or 2. Preferably, p and q are 0 or 1. In a particular embodiment, p and q are 0. In a further embodiment, p and q are 1.

R⁸ is hydrogen or C₁₋₄alkyl (for example, methyl). Preferably, R⁸ is hydrogen.

In an embodiment of the invention, R represents

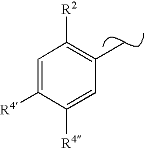

wherein R² is —NH—C(O)—COOH, —O—(C₁₋₄alkyl)-COOH, —C₁₋₄alkyl-COOH, or —COOH; and R^{4′} and R^{4″} each independently represent halogen or hydrogen. R^{4′} and R^{4″} may, for example, both be halogen (e.g. chlorine). Alternatively, for example, R⁴ may be halogen (e.g. chlorine) and R⁴¹ hydrogen.

In an embodiment of the invention, R represents

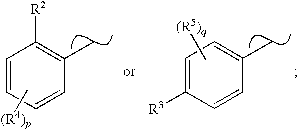

R² and R³ independently represent —NH—C(O)—COOH, —O—(C₁₋₄alkyl)-COOH, —C₁₋₄alkyl-COOH, or —COOH; each R⁴ and R⁵ independently represent halogen, CF₃ or C₁₋₄alkyl; p and q are independently 0, 1 or 2; R⁸ is hydrogen or C₁₋₄alkyl; and R¹ represents the group:

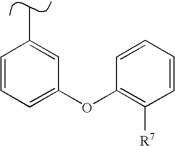

where R⁷ is hydrogen, methoxy or ethoxy.

In another embodiment, R represents

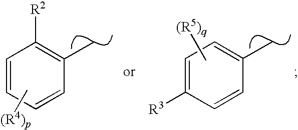

R² and R³ independently represent —NH—C(O)—COOH, —O—(C₁₋₄alkyl)-COOH, —C₁₋₄alkyl-COOH, or —COOH; each R⁴ and R⁵ independently represent halogen, CF₃ or C₁₋₄ alkyl; p and q are independently 0, 1 or 2; R⁸ is hydrogen or C₁₋₄alkyl; and R¹ represents the group:

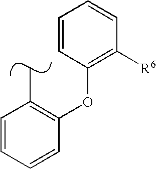

where R⁶ is independently hydrogen, methoxy or ethoxy.

In a further embodiment of the present invention, R represents

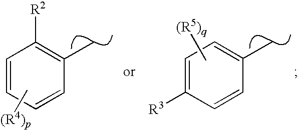

R² and R³ independently represent —NH—C(O)—COOH, —O—(C₁₋₄alkyl)-COOH, —C₁₋₄alkyl-COOH, or —COOH; each R⁴ and R¹ independently represent halogen; p and q are independently 0, 1 or 2; R⁸ is hydrogen; and R¹ represents the group:

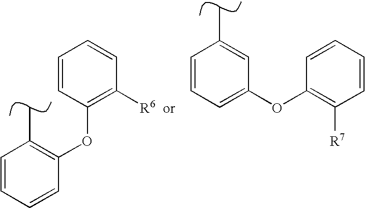

where R⁶ and R⁷ are independently hydrogen or methoxy.

For compounds of formula (II) which are capable of existing in stereoisomeric forms, it will be understood that the invention encompasses all geometric and optical isomers of the compounds of formula (II) and mixtures thereof including racemates. Tautomers and mixtures thereof also form an aspect of the present invention.

Preferred compounds of the present invention include:
(2-{[9-(2-phenoxybenzyl)-3,9-diazaspiro[5.5]undec-3-yl]carbonyl}phenyl)acetic acid,
(5-chloro-2-{[9-(2-phenoxybenzyl)-3,9-diazaspiro[5.5]undec-3-yl]carbonyl}phenyl)acetic acid,
(2-{[9-(3-phenoxybenzyl)-3,9-diazaspiro[5.5]undec-3-yl]carbonyl}phenyl)acetic acid,
[2-({9-[2-(2-methoxyphenoxy)benzyl]-3,9-diazaspiro[5.5]undec-3-yl}carbonyl)phenyl]acetic acid,
[5-chloro-2-({9-[2-(2-methoxyphenoxy)benzyl]-3,9-diazaspiro[5.5]undec-3-yl}carbonyl)phenyl]acetic acid,
4,5-dichloro-2-({9-[2-(2-methoxyphenoxy)benzyl]-3,9-diazaspiro[5.5]undec-3-yl}carbonyl)benzoic acid,
{[2-({9-[2-(2-methoxyphenoxy)benzyl]-3,9-diazaspiro[5.5]undec-3-yl}carbonyl)phenyl]amino}(oxo)acetic acid,
[2-({9-[3-(2-methoxyphenoxy)benzyl]-3,9-diazaspiro[5.5]undec-3-yl}carbonyl)phenyl]acetic acid,
2-({9-[3-(2-methoxyphenoxy)benzyl]-3,9-diazaspiro[5.5]undec-3-yl}carbonyl)benzoic acid,
[4-({9-[3-(2-methoxyphenoxy)benzyl]-3,9-diazaspiro[5.5]undec-3-yl}carbonyl)phenoxy]acetic acid,
[2-({9-[3-(2-methoxyphenoxy)benzyl]-3,9-diazaspiro[5.5]undec-3-yl}carbonyl)phenoxy]acetic acid,
   or a pharmaceutically acceptable salt thereof.

According to the present invention there is also provided a process for the preparation of compounds of formula (II) and salts thereof which comprises

(a) reacting a compound of formula (III):

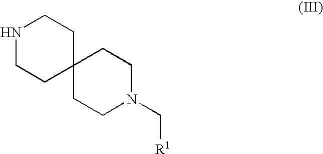

where R¹ is as defined in formula (II), with a compound of formula (IV):

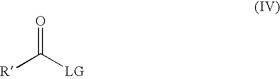

where LG is a suitable leaving group, and R′ is as defined in formula (II) but with the exception that R² is R^{2′} and R³ is R^{3′}, and wherein R^{2′} and R^{3′} independently represent —NH—C(O)—C(O)—OR″, O—(C₁₋₄alkyl)-C(O)—OR″, —C₁₋₄alkyl—C(O)—OR″, or —C(O)—OR″ where R″ is a suitable protecting group, e.g. C₁-C₆ alkyl (preferably methyl or ethyl), and thereafter removing the protecting group to form the corresponding acid functionality (e.g. where R² and R³ are ester groups, hydrolysing the ester groups R^{2′} and R^{3′} to the corresponding acid); or

(b) reaction of a compound of formula (V)

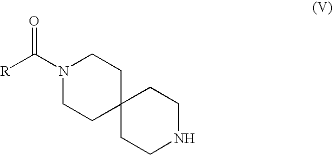

wherein R is as defined in formula (II) or is a protected derivative thereof, with an aldehyde compound of formula (VI):

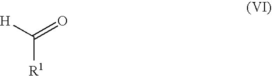

wherein R¹ is as defined in formula (II), or

(c) reaction of a compound of formula (V) defined above with a compound of formula (VII)

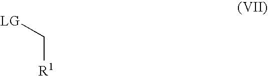

wherein R¹ is as defined in formula (II) and LG is a leaving group.

A compound of formula (III) can be prepared by process (d) by reacting a compound of formula (VIII)

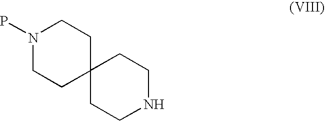

in which P is a protecting group, with a compound of formula (VI) as defined above, and subsequently removing the protecting group P.

A compound of formula (III) can also be prepared by process (e) by reacting a compound of formula (VIII) with a compound of formula (VII), and subsequently removing the protecting group P.

A compound of formula (V) can be prepared by process (f) by reacting a compound of formula (IX):

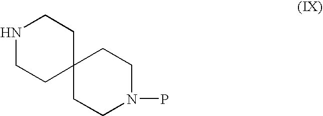

where P is a suitable protecting group with a compound of formula (IV) as defined above, and subsequently removing the protecting group P.

Process (a) may be carried out using standard coupling reactions that are well know in the art. A suitable leaving group LG is, for example OH or chlorine, preferably OH. The coupling reaction may typically carried out using activating reagents such as N-[(1H-1,2,3-benzotriazol-1-yloxy)(dimethylamino)methylene]-N-methylmethanaminium hexafluorophosphate (HBTU), N-[(dimethylamino)(3H-[1,2,3]triazolo[4,5-b]pyridin-3-yloxy)methylene]-N-methylmethanaminium hexafluorophosphate (HATU), or (benzotriazol-1-yloxy)tripyrrolidinophosphonium hexafluorophosphate (PYB OP). Typically, the reaction is carried out in the presence of a suitable base (e.g. triethylamine) and an organic solvent (e.g. dichloromethane) at a suitable temperature (e.g. room temperature). When R^{2′} and R³ are ester groups, de-esterification may be carried out in the presence of a base, e.g. LiOH.

Process (b) may be carried out via reductive amination, the procedures of which are well known in the art. Typically, the reaction is carried out in the presence of sodium triacetoxyborhydride [NaBH(OAc)₃]. Typically, the reaction is carried out in the presence of a suitable base (e.g. triethylamine) and an organic solvent (e.g. dichloromethane) at a suitable temperature (e.g. room temperature). In this process, group R may be protected by a suitable protecting group. As an example, the carboxyl functionality in R may be protected by being in the form of an ester group COOP′, where P′ is a suitable protecting group (e.g. methyl or ethyl). After the reaction, the protecting group can be removed to afford the carboxylic acid (e.g., in the case of an ester, hydrolysing the ester to afford the required acid functionality (or salt thereof)). However, a person skilled in the art would recognise that R may be protected by other functional groups (other than esters) which upon their removal, affords the required acid functionality (or salt thereof).

Process (c) may be carried out in a suitable organic solvent (e.g. DMF) at a suitable temperature (e.g. room temperature). The use of leaving groups are well known in the art for this type of reaction. Examples of typical leaving groups are halo, alkoxy, trifluoromethanesulfonyloxy, methanesulfonyloxy, or p-toluenesulfonyloxy. Typically, the leaving group is a halogen such as chlorine or bromine. The compound of formula (V) used in process (c) may protected as described above for process (b).

The coupling step of process (d) may be carried out according to the conditions described for process (b) above. The coupling step of process (e) may be carried out according to the conditions described for process (c) above. The coupling step of process (f) may be carried out according to the conditions described for process (a) above. The compound of formula (V) used in process (c) may be protected as described above for process (b). An example of a typical protecting group P used in processes (d), (e) and (f) is tert-butyloxycarbonyl (t-boc). However, other suitable protecting groups may be used.

The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991). After the coupling the protecting group P can be removed.

Compounds of formulae (IV), (VI), (VII), (VIII), and (IX) are either commercially available, are well known in the literature or may be prepared easily using known techniques, for example as shown in the accompanying Examples. US patent number U.S. Pat. No. 5,451,578 (Claremon et al.) describes, under example 1 of the patent, a process for synthesising tert-butyl 3,9-diazaspiro[5.5]undecane-3-carboxylate (corresponding to compound (IX) with P as tert-butyloxycarbonyl).

In so far as the intermediates referred to in the processes of the present invention are capable of forming salts, the processes of the invention described above encompass the use of the intermediates in salt form or free form.

The compounds of formula (II) above may be converted to a pharmaceutically acceptable salt thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate orp-toluenesulphonate.

The compounds of formula (II) and pharmaceutically acceptable salts thereof may exist in solvated, for example hydrated, as well as unsolvated forms, and the present invention encompasses all such solvated forms.

It will be appreciated that the compounds of formula (II) and salts thereof may exist as zwitterions and that while drawn in the acid form may exist also in internal salt (zwitterionic) form. The representation of formula (II) and the examples of the present invention covers both acid and zwitterionic forms and mixtures thereof in all proportions.

The compounds of formula (II) have activity as pharmaceuticals, in particular as modulators of chemokine receptor (especially CCR8) activity, and may be used in the treatment (therapeutic or prophylactic) of conditions/diseases in human and non-human animals which are exacerbated or caused by excessive or dysregulated production of chemokines. Examples of such conditions/diseases include:
(1) (the respiratory tract) obstructive airways diseases including chronic obstructive pulmonary disease (COPD), asthma, such as bronchial, allergic, intrinsic, extrinsic and dust asthma, particularly chronic or inveterate asthma (e.g. late asthma and airways hyper-responsiveness), bronchitis, acute, allergic, atrophic rhinitis and chronic rhinitis including rhinitis caseosa, hypertrophic rhinitis, rhinitis purulenta, rhinitis sicca and rhinitis medicamentosa, membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung and idiopathic interstitial pneumonia,
(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis,
(3) (skin) pruritis, scleroderma, otitus, psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis, lupus,
(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema,
(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia, polyneuropathies, e.g. Guillain-Barré syndrome, chronic inflammatory demyelinating polyradiculoneuropathy, multifocal motor neuropathy, plexopathies, CNS demyelination, e.g. multiple sclerosis, acute disseminated/haemorrhagic encephalomyelitis, and subacute sclerosing panencephalitis, neuromuscular disorders, e.g. myasthenia gravis and Lambert-Eaton syndrome, spinal disorders, e.g. tropical spastic paraparesis, and stiff-man syndrome: paraneoplastic syndromes, e.g. cerebellar degeneration and encephalomyelitis, CNS trauma, migraine, stroke and correctum diseases such as meningitis
(6) (other tissues and systemic disease) hepatitis, vasculitis, spondyloarthopathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura, post-operative adhesions, and sepsis.
(7) (allograft and xenograft rejection) acute and chronic following, for example, transplantation of kidney, heart, liver, lung, bone marrow, skin and cornea, and chronic graft versus host disease,
(8) Cancer, carcinoma & tumour metastasis, including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.
(9) All diseases that result from a general inbalance of the immune system and resulting in increased atopic inflammatory reactions.
(10) Cystic fibrosis, re-perfusion injury in the heart, brain, peripheral limbs and other organs.
(11) Burn wounds & chronic skin ulcers
(12) Reproductive Diseases (e.g. Disorders of ovulation, menstruation and implantation, Pre-term labour, Endometriosis)
(13) thrombosis
(14) infectious diseases such as HIV infection and other viral infections, bacterial infections.

Thus, the present invention provides a compound of formula (II) or a pharmaceutically-acceptable salt thereof, as hereinbefore defined for use in therapy.

In a still further aspect, the present invention provides the use of a compound of formula (II) or a pharmaceutically acceptable salt thereof, as hereinbefore defined in the manufacture of a medicament for the treatment of human diseases or conditions in which modulation of chemokine receptor activity, particularly CCR8 activity, is beneficial.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CCR8) receptor, which comprises administering to a patient a therapeutically effective amount of a compound of formula (II) or a pharmaceutically acceptable salt thereof.

The invention also provides a method of treating a respiratory disease, such as asthma, COPD or rhinitis, in a patient suffering from, or at risk of, said disease, which comprises administering to the patient a therapeutically effective amount of a compound of formula (II) or a pharmaceutically acceptable salt thereof, as hereinbefore defined.

For the above-mentioned therapeutic uses the dosage administered will, of course, vary with the compound employed, the mode of administration, the treatment desired and the disorder indicated.

The compounds of formula (II) and pharmaceutically acceptable salts thereof may be used on their own but will generally be administered in the form of a pharmaceutical composition in which the formula (II) compound or salt thereof (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (II) or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (II) or a pharmaceutically acceptable salt thereof, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations, or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention further relates to combination therapies wherein a compound of the invention or a pharmaceutically acceptable salts or solvate thereof, or a pharmaceutical composition or formulation comprising a compound of formula (II) is administered concurrently or sequentially with therapy and/or an agent for the treatment of any one of asthma, allergic rhinitis, cancer, COPD, rheumatoid arthritis, psoriasis, inflammatory bowel diseases, osteoarthritis or osteoporosis.

In particular, for the treatment of the inflammatory diseases rheumatoid arthritis, psoriasis, inflammatory bowel disease, COPD, astluna and allergic rhinitis the compounds of the invention may be combined with agents such as TNF-α inhibitors such as anti-TNF monoclonal antibodies (such as Remicade, CDP-870 and D₂E₇ and TNF receptor immunoglobulin molecules (such as Enbrel®), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide, ciclesonide, hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton, ABT-761, fenleuton, tepoxalin, Abbott-79175, Abbott-85761, N-(5-substituted)-thiophene-2-alkylsulfonamides, 2,6-di-tert-butylphenol hydrazones, methoxytetrahydropyrans such as Zeneca ZD-2138, the compound SB-210661, pyridinyl-substituted 2-cyanonaphthalene compounds such as L-739,010, 2-cyanoquinoline compounds such as L-746,530, indole and quinoline compounds such as MK-591, MK-886, and BAY x 1005.

The present invention still further relates to the combination of a compound of the invention together with a receptor antagonist for leukotrienes LTB₄, LTC₄, LTD₄, and LTE₄ selected from the group consisting of the phenothiazin-3-ones such as L-651,392, amidino compounds such as CGS-25019c, benzoxalamines such as ontazolast, benzenecarboximidamides such as BIIL 284/260, and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistaminic H₂ receptor antagonists such as cetirizine, loratadine, desloratadine, fexofenadine, astemizole, azelastine, and chlorpheniramine.

The present invention still further relates to the combination of a compound of the invention together with a gastroprotective H₂ receptor antagonist.

The present invention still further relates to the combination of a compound of the invention together with an α₁.- and α₂.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide, tiotropium bromide, oxitropium bromide, pirenzepine, and telenzepine.

The present invention still further relates to the combination of a compound of the invention together with a β₁- to β₄-adrenoceptor agonists such as metaproterenol, isoproterenol, isoprenaline, albuterol, salbutamol, formoterol, salmeterol, terbutaline, orciprenaline, bitolterol mesylate, and pirbuterol, or methylxanthanines including theophylline and aminophylline, sodium cromoglycate, or muscarinic receptor (M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase, especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family), CXCR1, CXCR2, CXCR3, CXCR4 and CXCR5 (for the C—X—C family) and CX₃CR1 for the C—X₃—C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of the invention together with (i) tryptase inhibitors, (ii) platelet activating factor (PAF) antagonists, (iii) interleukin converting enzyme (ICE) inhibitors, (iv) IMPDH inhibitors, (v) adhesion molecule inhibitors including VLA-4 antagonists, (vi) cathepsins, (vii) MAP kinase inhibitors, (viii) glucose-6 phosphate dehydrogenase inhibitors, (ix) kinin-B₁- and B₂-receptor antagonists, (x) anti-gout agents, e.g., colchicine, (xi) xanthine oxidase inhibitors, e.g., allopurinol, (xii) uricosuric agents, e.g., probenecid, sulfinpyrazone, and benzbromarone, (xiii) growth hormone secretagogues, (xiv) transforming growth factor (TGFβ), (xv) platelet-derived growth factor (PDGF), (xvi) fibroblast growth factor, e.g., basic fibroblast growth factor (bFGF), (xvii) granulocyte macrophage colony stimulating factor (GM-CSF), (xviii) capsaicin cream, (xix) Tachykinin NK₁ and NK₃ receptor antagonists selected from the group consisting of NKP-608C, SB-233412 (talnetant), and D-4418, (xx) elastase inhibitors selected from the group consisting of UT-77 and ZD-0892, (xxi) TNFα converting enzyme inhibitors (TACE), (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®), antitumour antibiotics (for example anthracyclines like adriamycin, bleomycin, doxorubicin, daunomycin, epirubicin, idarubicin, mitomycin-C, dactinomycin and mithramycin), antimitotic agents (for example vinca alkaloids like vincristine, vinblastine, vindesine and vinorelbine and taxoids like taxol and taxotere), and topoisomerase inhibitors (for example epipodophyllotoxins like etoposide and teniposide, amsacrine, topotecan and camptothecin),
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride,
(iii) Agents which inhibit cancer cell invasion (for example metalloproteinase inhibitors like marimastat and inhibitors of urokinase plasminogen activator receptor function),
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family,
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin),
(vi) vascular damaging agents such as Combretastatin A4 and compounds disclosed in International Patent Applications WO 99/02166, WO00/40529, WO 00/41669, WO01/92224, WO02/04434 and WO02/08213,
(vii) antisense therapies, for example those which are directed to the targets listed above, such as ISIS 2503, an anti-ras antisense,
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy, and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

### General Procedures

### HPLC Conditions

a. Method A

HPLC method A was performed with Agilent 1100 series machines on Kromassil© C18 5 μm 3.0×100 mm colum. Aqueous phase was water/TFA (99.8/0.1) and organic phase was acetonitrile/TFA (99.92/0.08). Flow was 1 mL/min and gradient was set from 10 to 100% of organic phase during 20 minutes. Detection was carried out on 220, 254 and 280 nm.

b. Method B

HPLC method B was performed with Agilent 1100 series machines on XTerra® RP₈ 5 μm 3.0×100 mm colum. Aqueous phase was 15 nM NH₃ in water and organic phase was acetonitrile. Flow was 1 mL/min and gradient was set from 10 to 100% of organic phase during 20 minutes. Detection was carried out on 220, 254 and 280 nm.

### STARTING MATERIALS FOR EXAMPLES 1 TO 11

### Intermediate A

### 3-(2-phenoxybenzyl)-3,9-diazaspiro[5.5]undecane Hydrochloride

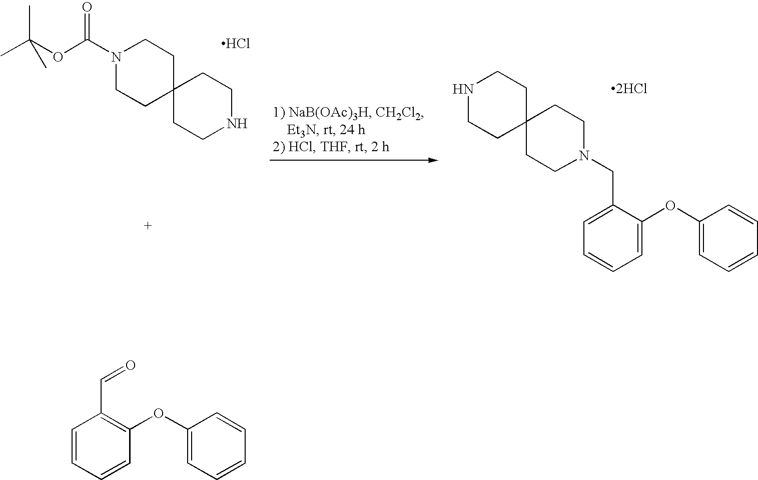

### a) tert-butyl 9-(2-phenoxybenzyl)-3,9-diazaspiro[5.5]undecane-3-carboxylate

tert-Butyl 3,9-diazaspiro[5.5]undecane-3-carboxylate hydrochloride (0.95 g, 3.26 mmol), 2-phenoxybenzaldehyde (0.70 g, 3.54 mmol) and sodium triacetoxyborohydride (0.97 g, 4.56 mmol) were stirred in a mixture of CH₂Cl₂ (20 mL), DMF (1.0 mL) and Et₃N (0.68 mL) for 24 h at room temperature. Na₂CO₃ (aq.sat) (30 mL) was added to the reaction mixture. The product was extracted with CH₂Cl₂, washed with water, dried and the solvent was evaporated. Column cromatography on SiO₂ with heptane/EtOAc 1:1 with 2% Et₃N gave the title compound (384 mg).

¹H NMR (400 MHz, CDCl₃): δ 7.52 (m, 1H), 7.29 (m, 3H), 7.14 (m, 1H), 7.06 (m, 1H), 6.91 (m, 3H), 3.59 (m, 2H), 3.35 (m, 4H), 1.45 (s, 9H), 1.69-1.32 (m, 4H)

### b) 3-(2-phenoxybenzyl)-3,9-diazaspiro[5.5]undecane hydrochloride

tertI-butyl 9-(2-phenoxybenzyl)-3,9-diazaspiro[5.5]undecane-3-carboxylate (0.873 g, 2.0 mmol) was dissolved in THF (50 mL) and HCl (conc.) (15 mL) and the reaction mixture was stirred for 2 h at room temperature. After evaporation of the solvent the residue was dissolved in toluene/MeOH 1:1 and evaporated to give the title compound as an white glass. (0.775 g).

APCI-MS, m/z 337 (MH⁺)

### Intermediate B

### 3-[3-phenoxybenzyl]-3,9-diazaspiro[5.5]undecane Dihydrochloride

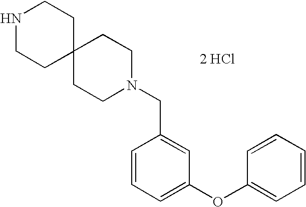

### a) tert-butyl 9-(3-phenoxybenzyl)-3,9-diazaspiro[5.5]undecane-3-carboxylate

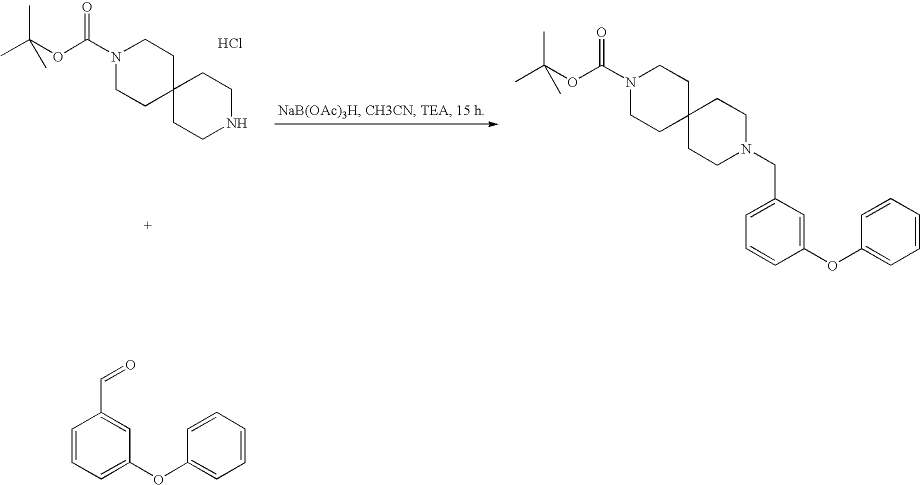

A mixture of tert-butyl 3,9-diazaspiro[5.5]undecane-3-carboxylate hydrochloride (1.0 g, 3.4 mmol), 3-phenoxybenzaldehyde (0.75 g, 3.8 mmol), triethylamine (0.72 mL, 5.2 mmol), sodium triacetoxyborohydride (1.02 g, 4.8 mmol), dichloroethane (35 mL) and dimethylformamide (5 mL) was heated at reflux overnight. The reaction mixture was partitioned between ethyl acetate and saturated sodium hydrogen carbonate solution. The organic layer was isolated and evaporated to dryness. Column cromatography on SiO₂ gave the title compound (0.71 g, 47%).

APCI-MS m/z: 437.3 [MH+]

### b) 3-(3-phenoxybenzyl)-3,9-diazaspiro[5.5]undecane Dihydrochloride

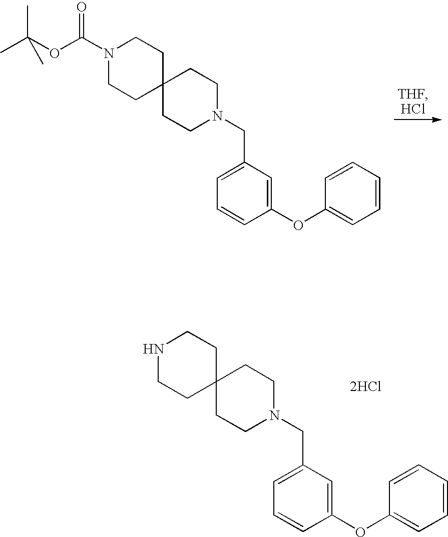

To a solution of tert-butyl 9-(3-phenoxybenzyl)-3,9-diazaspiro[5.5]undecane-3-carboxylate (0.71 g, 1.6 mmol) in 50 mL of THF was 5 mL of conc. HCl added. After 2 h of stirring at room temperature the reaction mixture was evaporated and co-evaporated three times with methanol and toluene. The title compound was obtained as a white solid.

APCI-MS m/z: 337.2 [MH+]

### Intermediate C

### 3-[2-(2-methoxyphenoxy)benzyl]-3,9-diazaspiro[5.5]undecane Dihydrochloride

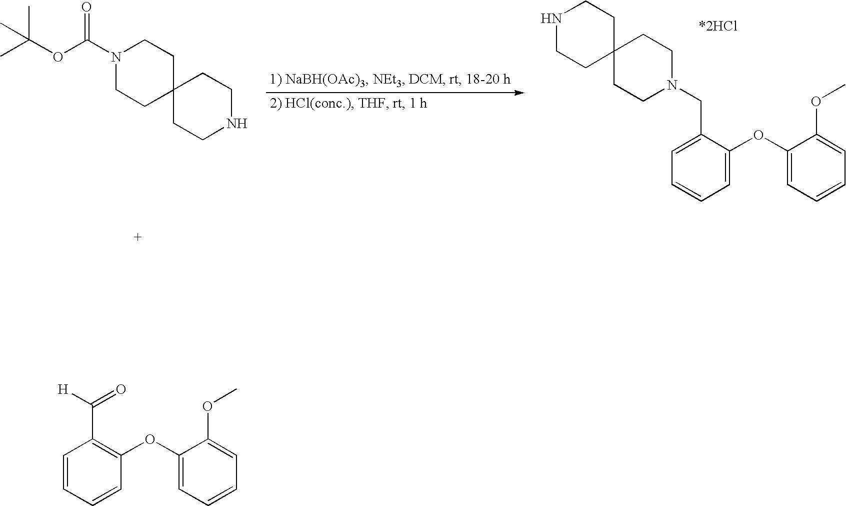

### a) tert-butyl 9-[2-(2-methoxyphenoxy)benzyl]-3,9-diazaspiro[5.5]undecane-3-carboxylate

tert-Butyl 3,9-diazaspiro[5.5]undecane-3-carboxylate hydrochloride (1.50 g, 5.2 mmol), 2-(2-methoxyphenoxy)benzaldehyde (1.24 g, 5.4 mmol), triethylamine (1.08 mL, 7.74 mmol) and sodium triacetoxyborohydride (1.23 g, 5.8 mmol) was dissolved in dichloromethane (40 mL) and dry DMF (15 mL). The pH was adjusted to 4 with AcOH and the mixture was stirred at room temperature over night. Another batch of sodium triacetoxyborohydride (1.0 g, 4.72 mmol) was added and the mixture was stirred at 40° C. for 2 hrs. The mixture was diluted with EtOAc (150 mL) and washed with sodium bicarbonate-solution, H₂O and brine and dried over Na₂SO₄ and evaporated. The crude product was purified using column chromatography on SiO₂ eluting with Heptane:EtOAc 4:1+2 vol % NEt₃ affording 1.27 g (53%) of the title compound as a colourless oil.

¹H NMR (400 MHz, DMSO-D6) δ 7.42 (dd, J=7.5, 1.5 Hz, 1H), 7.17-7.10 (m, 3H), 7.04 (td, J=7.4, 0.9 Hz, 1H), 6.95-6.88 (m, 2H), 6.84 (d, J=7.6 Hz, 1H), 6.58 (d, J=8.0 Hz, 1H), 3.74 (s, 3H), 3.54 (s, 2H), 3.30-3.23 (m, 6H), 2.40-2.34 (m, 4H), 1.46-1.40 (m, 12H), 1.38 (s, 11H), 1.34-1.29 (m, 14H)

APCI-MS m/z: 467.3 [MH+]

### b) 3-[2-(2-methoxyphenoxy)benzyl]-3,9-diazaspiro[5.5]undecane Dihydrochloride

The oil from part a) was dissolved in THF (100 mL) and conc. HCl (20 μL) was added and the mixture was stirred at room temperature for 1 hr. The solvents were evaporated and the crude product was evaporated twice with toluene and ethanol to remove traces of water, affording 1.59 g (quant.) of the title compound as a slightly purple oil. Some toluene (12 wt %) was still left in the compound, which did not disappear even after 24 hrs under vacuum.

¹H NMR (400 MHz, CD3OD) δ 7.57 (dd, J=7.6, 1.6 Hz, 1H), 7.38-7.27 (m, 2H), 7.24-7.08 (m, 7H (+toluene)), 7.05 (td, J=7.7, 1.4 Hz, 1H), 6.60 (d, J=8.3 Hz, 1H), 4.55 (s, 2H), 3.75 (s, 3H), 3.64-3.49 (m, 4H), 3.25-3.19 (m, 4H), 2.32 (s, 2H (toluene)), 2.06 (d, J=14.7 Hz, 2H), 1.95 (t, J=5.9 Hz, 2H), 1.89-1.62 (m, 6H)

APCI-MS m/z: 367.5 [MH+]

### Intermediate D

### 3-[3-(2-methoxyphenoxy)benzyl]-3,9-diazaspiro[5.5]undecane Dihydrochloride

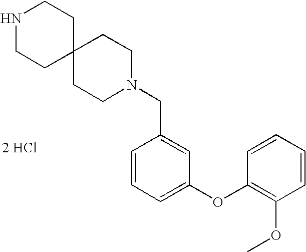

### a) 3-(2-methoxyphenoxy)benzaldehyde

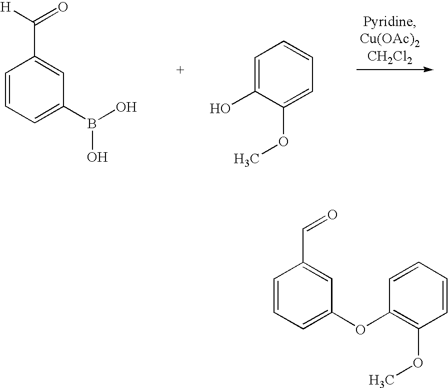

(3-Formylphenyl)boronic acid (5.0 g, 33 mmol) and guaiacol (2.8 g, 22 mmol) were mixed with Cu(OAc)₂ (4.0 g, 22 mmol), 4 Å molecular sieves and pyridine (9 mL) in dry dichloromethane (150 mL) and the resulting mixture was stirred overnight at room temperature. The reaction mixture was filtered and concentrated. Column cromatography on SiO₂ gave the title compound as an oil (1.7 g, 23%).

¹H NMR (400 MHz, CDCl₃) δ 9.95 (s, 1H), 7.58-7.54 (m, 1H), 7.47 (t, J=7.8 Hz, 1H), 7.38-7.34 (m, 1H), 7.26-7.19 (m, 2H), 7.08-7.02 (m, 2H), 7.01-6.95 (m, 1H), 3.82 (s, 3H)

GC-MS m/z: 228.0 [M]

### b) tert-butyl 9-[3-(2-methoxyphenoxy)benzyl]-3,9-diazaspiro[5.5]undecane-3-carboxylate

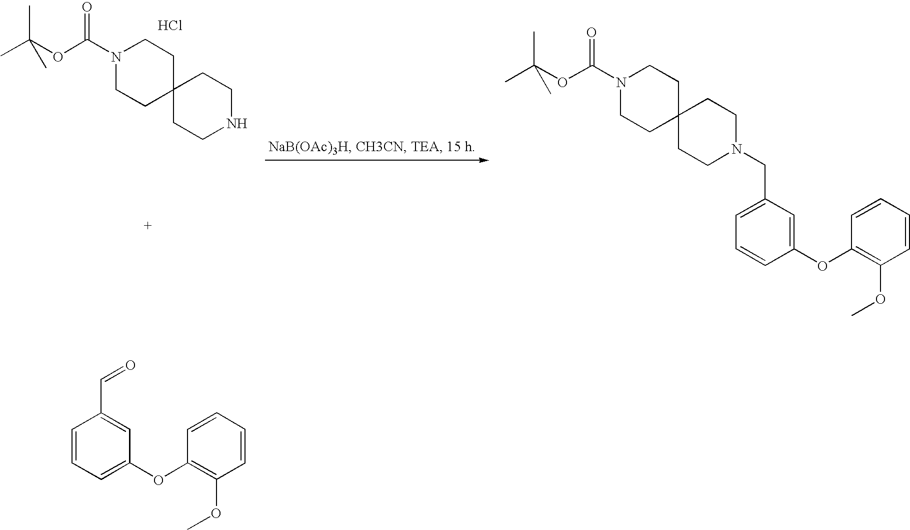

A mixture of tert-butyl 3,9-diazaspiro[5.5]undecane-3-carboxylate hydrochloride (1.4 g, 5.0 mmol), 3-(2-methoxyphenoxy)benzaldehyde (1.7 g, 7.5 mmol, triethylamine (1 mL, 7.5 mmol), sodium triacetoxyborohydride (1.6 g, 7.5 mmol) and acetonitrile were heated at reflux overnight. The reaction mixture was partitioned between ethyl acetate and saturated sodium hydrogen carbonate solution. The organic layer was isolated and evaporated to dryness. Column cromatography on SiO₂ gave the title compound (1.5 g, 64%).

¹H NMR (400 MHz, DMSO-D6) δ 7.26-7.14 (m, 3H), 7.04-6.90 (m, 3H), 6.76 (s, 1H), 6.71-6.66 (m, 1H), 3.39 (s, 2H), 3.31 (s, 5H), 3.29-3.23 (m, 4H), 2.33-2.25 (m, 4H), 1.43-1.36 (m, 11H), 1.35-1.27 (m, 4H)

APCI-MS m/z: 467.3 [MH+]

### c) 3-[3-(2-methoxyphenoxy)benzyl]-3,9-diazaspiro[5.5]undecane Dihydrochloride

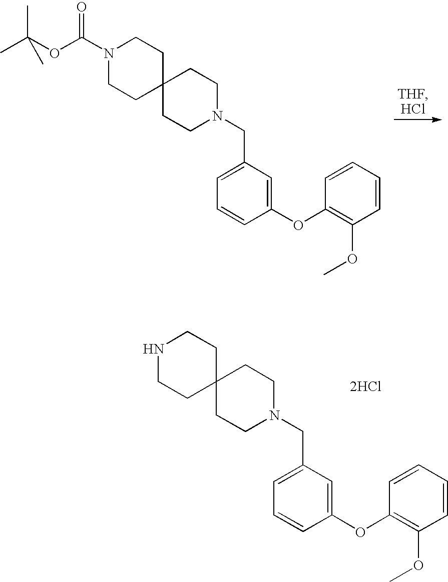

To a solution of tert-butyl 9-[3-(2-methoxyphenoxy)benzyl]-3,9-diazaspiro[5.5]undecane-3-carboxylate (1.6 g, 3.4 mmol) in 50 mL of THF was added 7 mL of conc. HCl. After 2 h stirring at room temperature the reaction mixture was evaporated and co-evaporated three times with methanol and toluene. The title compound was obtained as a white solid.

¹H NMR (400 MHz, DMSO-D6) δ 7.37 (t, J=7.9 Hz, 1H), 7.29 (d, J=7.7 Hz, 1H), 7.26-7.16 (m, 2H), 7.14 (s, 1H), 7.10-7.05 (m, 1H), 7.02-6.96 (m, 1H), 6.88-6.81 (m, 1H), 4.25 (d, J=5.4 Hz, 2H), 3.73 (s, 3H), 3.13-2.94 (m, 8H), 1.88-1.64 (m, 6H), 1.56-1.47 (m, 2H)

APCI-MS m/z: 367.2 [MH+]

### Example 1

### (2-{[9-(2-phenoxybenzyl)-3,9-diazaspiro[5.5]undec-3-yl]carbonyl}phenyl)acetic Acid

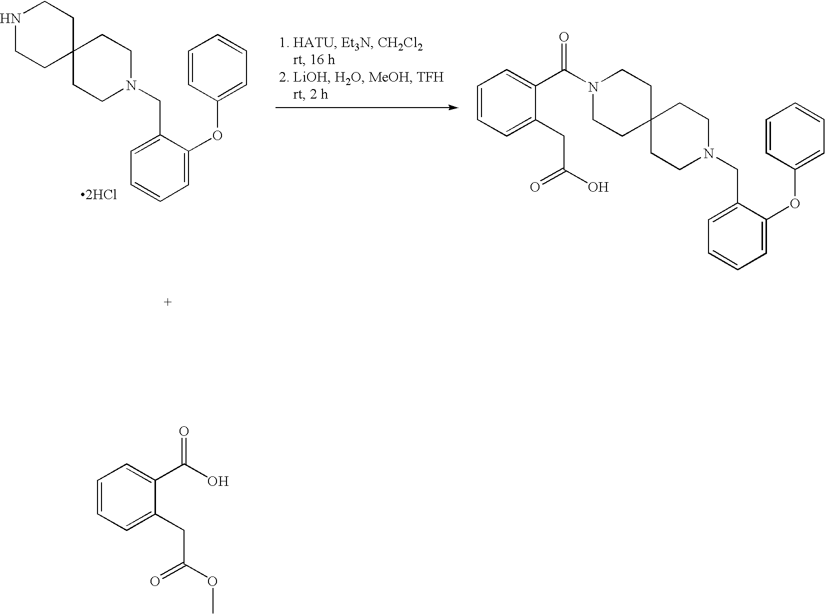

A mixture of intermediate A (88 mg, 0.20 mmol), 2-(2-methoxy-2-oxoethyl)benzoic acid (47 mg, 0.24 mmol), triethylamine (200 μl, 1.4 mmol), N—[(dimethylamino)(3H-[1,2,3]triazolo[4,5-b]pyridin-3-yloxy)methylene]-N-methylmethanaminium hexafluorophosphate (HATU, 91 mg, 0.24 mmol) and dichloromethane (2 mL) was stirred at ambient temperature for 1 h and then evaporated. The crude methyl ester was purified by column chromatography (SiO₂, gradient EtOAc/MeOH/Et₃N 100/0/0 to 79/20/1) and LiOH (44 mg, 1.8 mmol), THF (2 mL), MeOH (1 mL) and water (1 mL) were added to the evaporated fractions containing the intermediate ester. The mixture was stirred at ambient temperature for 2 h, acetic acid (1 mL) was added and the product was purified with preparative HPLC (RP-18, gradient acetonitrile/water/NH4OAc 10/90/0.1) to 95/5/0.1) to give the title compound as a white solid (45 mg, 45%).

¹H NMR (400 MHz, CD3OD with NaOD added): δ 7.47 (d, J=6.4 Hz, 1H), 7.41-7.22 (m, 6H), 7.15 (t, J=7.2 Hz, 2H), 7.06 (s, 1H), 6.90-6.85 (m, 3H), 3.92-3.80 (m, 1H), 3.61-3.38 (m, 5H), 3.28-3.21 (m, 2H), 2.56-2.40 (m, 4H), 1.69-1.26 (m, 8H)

APCI-MS m/z: 499.3 [MH+]

HPLC (Method A) RT: 7.88 min

### Example 2

### (5-chloro-2-{[9-(2-phenoxybenzyl)-3,9-diazaspiro[5.5]undec-3-yl]carbonyl}phenyl)acetic Acid

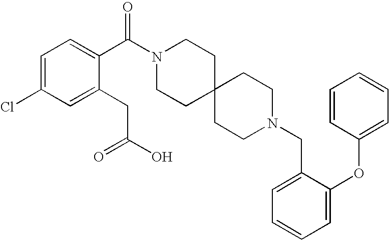

The title compound was prepared using the procedure of Example 1, but using intermediate A and 4-chloro-2-[2-methoxy-1-(methoxycarbonyl)-2-oxoethyl]benzoic acid as starting materials. Hydrolysis of the intermediate dimethyl (5-chloro-2-{[9-(2-phenoxybenzyl)-3,9-diazaspiro[5.5]undec-3-yl]carbonyl}phenyl)malonate was performed at 50° C. to give the product as a white solid.

¹H NMR (400 MHz, CD3OD with NaOD added): δ 7.59 (dd, 1H), 7.45-7.37 (m, 4H), 7.27 (dd, 1H), 7.20 (dd, 2H), 7.14 (d, 1H), 7.09-7.02 (m, 2H), 6.87 (d, 1H), 4.30 (s, 2H), 3.73 (d, 2H), 3.52 (dd, 2H), 3.28-3.12 (m, 6H), 1.85-1.30 (m, 8H)

APCI-MS m/z: 542.9 [MH+]

HPLC (Method A) RT: 9.00 min

HPLC (Method B) RT: 5.95 min

### Example 3

### (2-{[9-(3-phenoxybenzyl)-3,9-diazaspiro[5.5]undec-3-yl]carbonyl}phenyl)acetic Acid

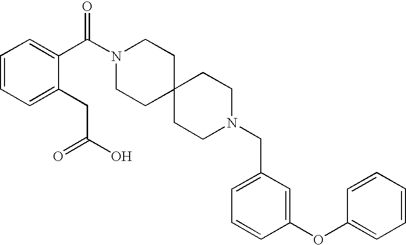

The title compound was prepared using the procedure of Example 1, but using intermediate B and 2-(2-methoxy-2-oxoethyl)benzoic acid as starting materials, to give the product as a white solid.

¹H NMR (400 MHz, CD3OD with NaOD added): δ 7.59 7.43-7.22 (m, 6H), 7.19-7.04 (m, 3H), 6.99-6.93 (m, 3H), 6.90-6.85 (m, 1H), 3.92-3.81 (m, 1H), 3.64-3.38 (m, 5H), 3.29-3.23 (m, 2H), 2.51-2.35 (m, 4H), 1.68-1.31 (m, 8H)

APCI-MS m/z: 449.7 [MH+]

HPLC (Method A) RT: 8.02 min

### Example 4

### [2-({9-[2-(2-methoxyphenoxy)benzyl]-3,9-diazaspiro[5.5]undec-3-yl}carbonyl)phenyl]acetic Acid

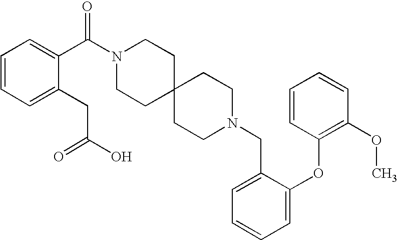

The title compound was prepared using the procedure of Example 1 but using intermediate C and 2-(2-methoxy-2-oxoethyl)benzoic acid as starting materials, to give the product as a white solid.

¹H NMR (400 MHz, CD3OD with NaOD added): δ 7.40 (t, 2H), 7.33 (t, 1H), 7.24 (t, 1H), 7.18-7.08 (m, 3H), 7.02 (t, 1H), 6.96-6.86 (m, 2H), 6.59 (d, 1H), 3.92-3.82 (m, 1H), 3.76 (s, 3H), 3.69 (s, 2H), 3.63-3.39 (m, 3H), 3.29-3.23 (m, 2H), 2.63-2.50 (m, 4H), 1.68-1.31 (m, 8H)

APCI-MS m/z: 529.3 [MH+]

HPLC (Method A) RT: 8.10 min

HPLC (Method B) RT-5.09 min

### Example 5

### [5-chloro-2-({9-[2-(2-methoxyphenoxy)benzyl]-3,9-diazaspiro[5.5]undec-3-yl}carbonyl)phenyl]acetic Acid

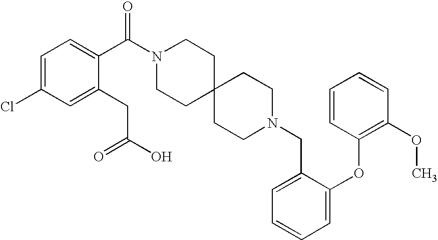

The title compound was prepared using the procedure of Example 1 but using intermediate C and 4-chloro-2-[2-methoxy-1-(methoxycarbonyl)-2-oxoethyl]benzoic acid as starting materials, to give the product as a white solid.

¹H NMR (400 MHz, CD3OD with NaOD added): δ 7.40 (t, J=7.1 Hz, 1H), 7.33 (t, J=7.5 Hz, 1H), 7.24 (t, J=7.4 Hz, 1H), 7.18-7.07 (m, 4H), 7.02 (t, J=7.4 Hz, 1H), 6.95-6.86 (m, 2H), 6.59 (d, J=8.2 Hz, 1H), 3.92-3.83 (m, 1H), 3.76 (s, 3H), 3.69 (s, 2H), 3.63-3.39 (m, 3H), 3.29-3.23 (m, 2H), 2.64-2.49 (m, 4H), 1.70-1.44 (m, 7H), 1.40-1.30 (m, 1H)

APCI-MS m/z: 563.5 [MH+]

HPLC (Method A) RT: 8.68 min

HPLC (Method B) RT: 5.34 min

### Example 6

### 4,5-dichloro-2-({9-[2-(2-methoxyphenoxy)benzyl]-3,9-diazaspiro[5.5]undec-3-yl}carbonyl)benzoic Acid

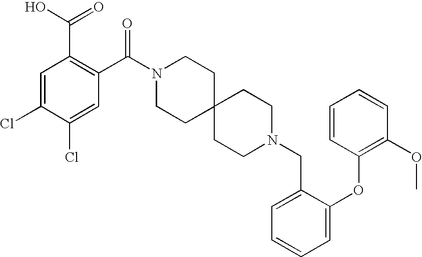

A mixture of intermediate C (88 mg, 0.20 mmol), 5,6-dichloro-2-benzofuran-1,3-dione (48 mg, 0.22 mmol), triethylamine (100 μl, 0.70 mmol) and dichloromethane (1 mL) was stirred at ambient temperature for 1 h, then evaporated and the product was purified with preparative HPLC(RP-18, gradient acetonitrile/water/NH4OAc 10/90/0.1) to 95/5/0.1) to give the product as a white solid (97 mg).

¹H NMR (400 MHz, CD3OD): δ 7.99 (s, 1H), 7.36-7.26 (m, 2H), 7.16 (dd, 1H), 7.11-7.05 (m, 2H), 7.01-6.83 (m, 4H), 4.14 (s, 2H), 3.73-3.53 (m, 5H), 3.27-3.00 (m, 6H), 1.82-1.27 (m, 8H)

APCI-MS m/z: 583.4 [MH+]

HPLC (Method A) RT: 9.54 min

HPLC (Method B) RT: 7.22 min

### Example 7

{[2-({9-[2-(2-methoxyphenoxy)benzyl]-3,9-diazaspiro[5.5]undec-3-yl}carbonyl)phenyl]amino}(oxo)acetic Acid

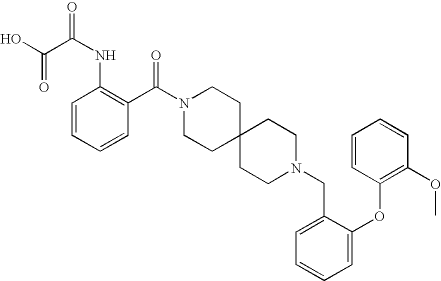

The amide coupling procedure of Example 1 using intermediate C and 2-aminobenzoic acid was used to prepare [2-({9-[2-(2-methoxyphenoxy)benzyl]-3,9-diazaspiro[5.5]undec-3-yl}carbonyl)phenyl]amine. To this aniline (110 mg, 0.22 mmol), were added methyl chloro(oxo)acetate (30 μL, 0.32 mmol), triethylamine (40 μL, 0.29 mmol) and THF (2 mL). After 30 minutes at ambient temperature water (2 mL), methanol (1 mL) and lithium hydroxide (120 mg, 5 mmol) were added and mixture was stirred at 50° C. for 2 hrs. By addition of acetic acid and ammonia the pH was adjusted to 5 and the product was purified with preparative HPLC (RP-18, gradient acetonitrile/water/NH4OAc 10/90/0.1) to 95/5/0.1) to give the product as a white solid (31 mg, 28%).

¹H NMR (400 MHz, DMSO-D6): δ 10.12 (s, 1H), 8.19 (s, 1H), 7.58 (d, J=7.1 Hz, 1H), 7.39 (t, J=9.2 Hz, 1H), 7.34-7.17 (m, 4H), 7.16-7.05 (m, 3H), 7.01 (t, J=7.6 Hz, 1H), 6.52 (d, J=8.1 Hz, 1H), 4.49-4.22 (m, 2H), 3.76-3.47 (m, 5H), 3.25-2.90 (m, 6H), 1.89-1.03 (m, 8H)

APCI-MS nm/z: 557.9 [MH+]

HPLC (Method A) RT: 7.64 min

HPLC (Method B) RT: 5.63 min

### Example 8

### [2-({9-[3-(2-methoxyphenoxy)benzyl]-3,9-diazaspiro[5.5]undec-3-yl}carbonyl)phenyl]acetic Acid

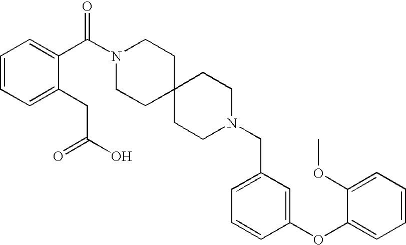

The title compound was prepared using the procedure of Example 1 but using intermediate D and 2-(2-methoxy-2-oxoethyl)benzoic acid as starting materials, to give the product as a white solid.

¹H NMR (400 MHz, CD3OD with NaOD added): 7.40 (d, J=1H), 7.34 (t, J=1H), 7.28-7.08 (m, 5H), 7.01-6.92 (m, 3H), 6.83 (s, 1H), 6.75 (d, 1H), 3.93-3.83 (m, 1H), 3.86 (s, 3H), 3.62-3.40 (m, 5H), 3.29-3.24 (m, 2H), 2.50-2.34 (m, 4H), 1.68-1.45 (m, 7H), 1.40-1.30 (m, 1H)

APCI-MS m/z: 529.5 [MH+]

HPLC (Method A) RT: 7.74 min

### Example 9

### 2-({9-[3-(2-methoxyphenoxy)benzyl]-3,9-diazaspiro[5.5]undec-3-yl}carbonyl)benzoic Acid

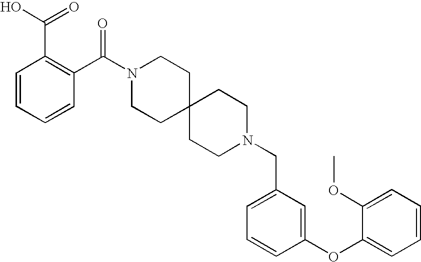

The title compound was prepared using the procedure of Example 6 but using intermediate D and 2-benzofuran-1,3-dione as starting materials, to give the product as a white solid.

¹H NMR (400 MHz, DMSO-D6): δ 9.14-8.98 (m, 1H), 7.84 (d, 1H), 7.60-7.52 (m, 1H), 7.39-6.98 (m, 9H), 6.55-6.48 (m, 1H), 4.45 (d, 2H), 3.75-3.66 (m, 3H), 3.64-3.45 (m, 2H), 3.30-2.92 (m, 4H), 1.97-1.05 (m, 10H)

APCI-MS m/z: 515.9 [MH+]

HPLC (Method A) RT: 7.55 min

HPLC (Method B) RT: 5.79 min

### Example 10

### [4-({9-[3-(2-methoxyphenoxy)benzyl]-3,9-diazaspiro[5.5]undec-3-yl}carbonyl)phenoxy]acetic Acid

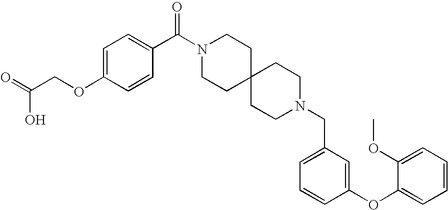

The title compound was prepared using the procedure of Example 1 but using intermediate D and 4-(2-methoxy-2-oxoethoxy)benzoic acid as starting materials, to give the product as a white solid.

APCI-MS m/z: 544.9 [MH+]

HPLC (Method A) RT: 7.57 min

### Example 11

### [2-({9-[3-(2-methoxyphenoxy)benzyl]-3,9-diazaspiro[5.5]undec-3-yl}carbonyl)phenoxy]acetic Acid

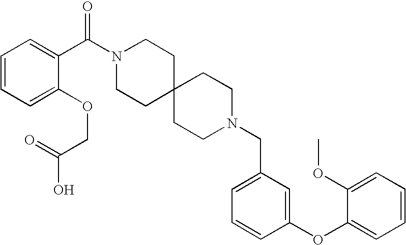

The title compound was prepared using the procedure of Example 1 but using intermediate D and 2-(2-methoxy-2-oxoethoxy)benzoic acid as starting materials, to give the product as a white solid.

¹H NMR (400 MHz, CD3OD with NaOD added): δ 7.36 (s, 1H), 7.26-7.15 (m, 3H), 7.11 (d, J=7.9 Hz, 1H), 7.04-6.91 (m, 5H), 6.83 (s, 1H), 6.76 (s, 1H), 4.43 (d, J=21.0 Hz, 2H), 3.81-3.66 (m, 5H), 3.47 (s, 2H), 3.42-3.22 (m, 2H), 2.49-2.35 (m, 4H), 1.66-1.46 (m, 7H), 1.43-1.33 (m, 1H)

APCI-MS m/z: 544.9 [MH+]

HPLC (Method A) RT: 8.02 min

HPLC (Method B) RT: 5.57 min

### Pharmacological Data

### CCL1 SPA Binding Assay

Membranes from CHO-K1 cells transfected with human recombinant chemokine CCR8 receptor (ES-136-M) were purchased from Euroscreen. Membrane preparations are stored at −70C in 7.5 mM Tris-Cl pH 7.5, 12.5 mM MgCl₂, 0.3 mM EDTA, 1 mM EGTA, 250 mM sucrose until used.

The CCR8 membranes (50.6 mg/ml) were preincubated with Wheat Germ Agglutinin SPA beads (4.05 mg/ml) in assay buffer (50 mM HEPES, 1 mM CaCl₂x2H₂O, 5 mM MgCl₂x6H₂O, 75 mM NaCl, 0.1% BSA) at pH=7.4 for 2 hours on ice. A 10-point dose-response curve (final concentrations 50 μM, 16.7 μM, 5.6 μM, 1.9 μM, 0.62 μM, 0.21 μM, 0.069 μM, 0.023 μM) was prepared by diluting compounds by serial dilution 1:3 in DMSO. In the screening plate (Polystyrene NBS plates, Costar Corning 3604) 1 μl from the DMSO solutions of compounds was transferred into each well. 1 μl of DMSO was added to the blank control wells and 1 μl unlabeled CCL1 (300 mM) was added to background control wells. 50 μl of the SPA bead-membrane mixture was added into each well. Finally, 50 μl (30 μM) ¹²⁵I CCL1 (2000 Ci/mM) was added to each well. Plates were then incubated at RT with shaking (700 rpm) for 90 minutes followed by 30 minutes at RT without shaking. The plate was read in a Wallac MicroBeta counter for 2 minutes/well.

### Human Microsomal Stability Assay

The assay is run in a 96-deepwell format at 1 mg microsomal protein (Xenotech)/mL in potassium phosphate buffer (pH 7.4) with a compound concentration of 2.5 μM and a NADPH concentration of 2 mM. Samples at four time-points (0, 5, 15 and 30 minutes) are withdrawn and the enzymatic reaction is terminated by protein precipitation with 1% acetic acid in acetonitrile. The incubations are performed on a thermostated plate (37° C.) placed on a Tecan worktable, and all liquid handling was performed robotically. After centrifugation of the samples, the supernatants are pooled in sets of four before they are analysed by liquid chromatography with tandem mass spectrometry detection (LC/MS/MS) using multiple reaction monitoring (MRM). Data are presented as intrinsic Clearance (CLᵢₙₜ), μl/min/mg protein, calculated from the initial linear part of the compound disappearance curve.

Tables 1 and 2 show the results that were obtained when the compounds of Examples 1 to 11 above, were tested in the above-described CCL1 SPA binding assay (expressed as IC50 values) and human microsomal stability assay. Data is also shown for four comparison compounds (A, B, X and Y)

Comparative examples A, B, X and Y are the following:

### A: (2-{[9-(2-isobutoxybenzyl)-3,9-diazaspiro[5.5]undec-3-yl]carbonyl}phenyl)acetic Acid

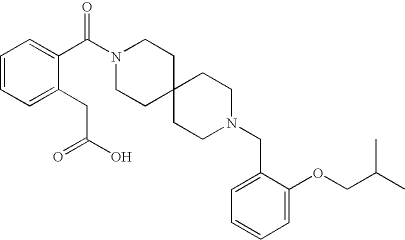

### B: 2-{[9-(2-isobutoxybenzyl)-3,9-diazaspiro[5.5]undec-3-yl]carbonyl}benzoic Acid

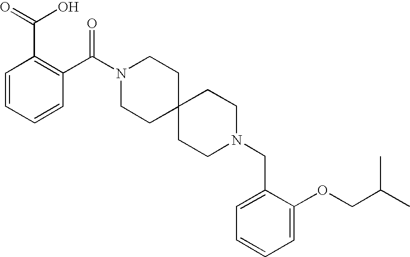

### X: 3-(4-chlorobenzoyl)-9-[2-(2-methoxyphenoxy)benzyl]-3,9-diazaspiro[5.5]undecane

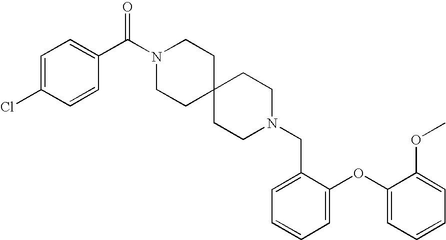

### Y: 3-(4-chlorobenzoyl)-9-[3-(2-methoxyphenoxy)benzyl]-3,9-diazaspiro[5.5]undecane

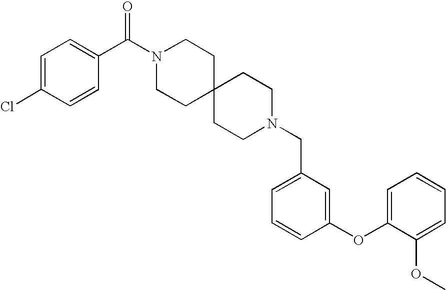

TABLE 1

| Example No. | IC50 (μM) |
|---|---|
| A | 1.06 |
| B | 1.37 |
| 1 | 0.643 |
| 2 | 0.196 |
| 3 | 0.144 |
| 4 | 0.058 |
| 5 | 0.017 |
| 6 | 0.081 |
| 7 | 0.100 |
| 8 | 0.042 |
| 9 | 0.071 |
| 10 | 0.405 |
| 11 | 0.520 |

TABLE 2

| Example No. | Intrinsic clearance (μl/min/mg) |
|---|---|
| 1 | <10 |
| 2 | <10 |
| 3 | <10 |
| 4 | <10 |
| 5 | <10 |
| 6 | 16 |
| 7 | <10 |
| 8 | <10 |
| 9 | <10 |
| 10 | <10 |
| 11 | <10 |
| X | >200 |
| Y | >200 |

1. A compound of formula: 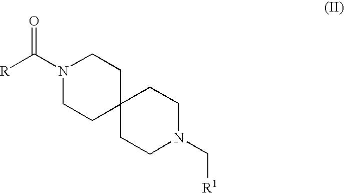
wherein R represents 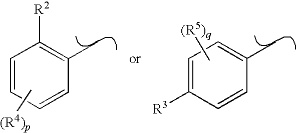
wherein
R² and R³ independently represent —NR⁸—C(O)—COOH, —O—(C₁₋₄alkyl)-COOH, —C₁₋₄alkyl-COOH, or —COOH;
each R⁴ and R⁵ independently represent halogen, CF₃ or C₁₋₄alkyl;
p and q are independently 0, 1 or 2;
R⁸ represents hydrogen or C₁₋₄alkyl;
R¹ represents the group: 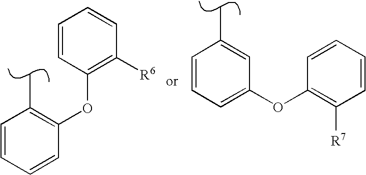
and R⁶ and R⁷ are independently hydrogen, methoxy or ethoxy;
or a pharmaceutically acceptable salt thereof.

2. The compound according to claim 1, wherein R is 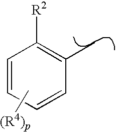
and wherein R², R⁴ and p are as defined in claim 1.

3. The compound according to claim 1, wherein R⁶ and R⁷ independently represent methoxy.

4. The compound according to claim 1, wherein R⁴ and R⁵ independently represent halogen.

5. The compound according to claim 1, wherein R² and R³ independently represent —NH—C(O)—COOH, —O—CH₂—COOH, or —CH₂—COOH.

6. The compound according to claim 1, wherein R² and R³ independently represent —CH₂—COOH.

7. The compound of formula (II) as defined in claim 1 being selected from the following or a pharmaceutically acceptable salt thereof:
(2-{[9-(2-phenoxybenzyl)-3,9-diazaspiro[5.5]undec-3-yl]carbonyl}phenyl)acetic acid,
(5-chloro-2-{[9-(2-phenoxybenzyl)-3,9-diazaspiro[5.5]undec-3-yl]carbonyl}phenyl)acetic acid,
(2-{[9-(3-phenoxybenzyl)-3,9-diazaspiro[5.5]undec-3-yl]carbonyl}phenyl)acetic acid,
[2-({9-[2-(2-methoxyphenoxy)benzyl]-3,9-diazaspiro[5.5]undec-3-yl}carbonyl)phenyl]acetic acid,
[5-chloro-2-({9-[2-(2-methoxyphenoxy)benzyl]-3,9-diazaspiro[5.5]undec-3-yl}carbonyl)phenyl]acetic acid,
4,5-dichloro-2-({9-[2-(2-methoxyphenoxy)benzyl]-3,9-diazaspiro[5.5]undec-3-yl}carbonyl)benzoic acid,
{[2-({9-[2-(2-methoxyphenoxy)benzyl]-3,9-diazaspiro[5.5]undec-3-yl}carbonyl)phenyl]amino}(oxo)acetic acid,
[2-({9-[3-(2-methoxyphenoxy)benzyl]-3,9-diazaspiro[5.5]undec-3-yl}carbonyl)phenyl]acetic acid,
2-({9-[3-(2-methoxyphenoxy)benzyl]-3,9-diazaspiro[5.5]undec-3-yl}carbonyl)benzoic acid,
[4-({9-[3-(2-methoxyphenoxy)benzyl]-3,9-diazaspiro[5.5]undec-3-yl}carbonyl)phenoxy]acetic acid, and
[2-({9-[3-(2-methoxyphenoxy)benzyl]-3,9-diazaspiro[5.5]undec-3-yl}carbonyl)phenoxy]acetic acid.

8. A pharmaceutical composition comprising a compound of formula (II), or a pharmaceutically acceptable salt thereof, as claimed in claim 1, in association with a pharmaceutically acceptable adjuvant, diluent or carrier.

9. The process for the preparation of a pharmaceutical composition as claimed in claim 8 which comprises mixing a compound of formula (II) or a pharmaceutically acceptable salt thereof, as claimed in claim 1 with a pharmaceutically acceptable adjuvant, diluent or carrier.

10-15. (canceled)

16. A method of treating a chemokine mediated disease wherein the chemokine binds to one or more chemokine receptors, which comprises administering to a patient a therapeutically effective amount of a compound of formula (II) or a pharmaceutically-acceptable salt thereof as claimed in claim 1.

17. The method according to claim 16 in which the chemokine receptor is the CCR⁸ receptor.

18. A method of treating a respiratory disease, which method comprises administering to a patient a therapeutically effective amount of a compound of formula (II) or a pharmaceutically-acceptable salt thereof as claimed in claim 1.

19. The method according to claim 18, wherein the respiratory disease is selected from the group consisting of asthma and chronic obstructive pulmonary disease.

20. A process for the preparation of a compound of formula (II) or a salt thereof as defined in claim 1 which comprises
(a) reacting a compound of formula (III): 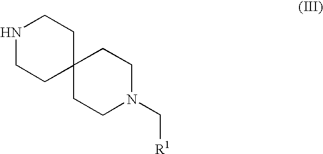
where R¹ is as defined in formula (II), with a compound of formula (IV): 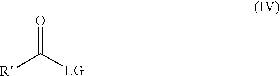
where LG is a suitable leaving group, and R¹ is as defined in formula (II) but with the exception that R² is R² and R³ is R³, and wherein R² and R³ independently represent —NH—C(O)—C(O)—OR″, O—(C₁₋₄alkyl)-C(O)—OR″, —C₁₋₄alkyl—C(O)—OR″, or —C(O)—OR″ where R″ is a suitable protecting group, and thereafter removing the protecting group to form the corresponding acid functionality; or
(b) reaction of a compound of formula (V) 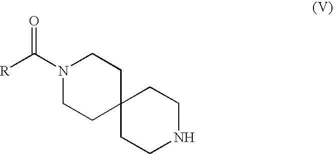
wherein R is as defined in formula (II) or is a protected derivative thereof, with an aldehyde compound of formula (VI): 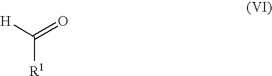
wherein R¹ is as defined in formula (II), or
(c) reaction of a compound of formula (V) defined (b) above with a compound of formula (VII) 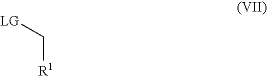
wherein R¹ is as defined in formula (II) and LG is a leaving group.
